(19) United States
(12) Patent Application Publication
(10) Pub. No.: US 2016/0051701 A1
(43) Pub. Date: Feb. 25, 2016

(54) IMPROVED METHODS FOR OSTEOARTHRITIS THERAPY

(71) Applicants: CELL-INNOVATIONS IP PTY LTD, Liverpool, New South Wales (AU); RUSTY PROPERTY HOLDINGS PTY LTD, Liverpool, New South Wales (AU)

(72) Inventors: Ralph Bright, Liverpool, New South Wales (AU); Wayne Thomas, Liverpool, New South Wales (AU)

(21) Appl. No.: 14/780,343

(22) PCT Filed: Mar. 28, 2014
(86) PCT No.: PCT/AU2014/000330

§ 371 Date: Sep. 25, 2015

(30) Foreign Application Priority Data
Mar. 28, 2013 (AU) 2013901078

(51) Int. Cl. A61K 49/00

(52) U.S. Cl. CPC A61K 49/0004

(57) ABSTRACT

The present invention provides improved methods for osteoarthritis therapy. In particular, the present invention provides a method for predicting the responsiveness of a subject to cell therapy for osteoarthritis comprising administering to the subject platelet-rich plasma (PRP) and assessing pain and/or mobility, wherein a decrease in pain and/or an increase in mobility indicates that the subject will be responsive to cell therapy.

### CROSS REFERENCE TO RELATED APPLICATIONS

This application claims priority from Australian Provisional Application No 2013901078 filed 28 Mar. 2013, the contents of which are incorporated herein by reference in their entirety.

### FIELD OF THE INVENTION

The present invention relates to improved methods for osteoarthritis therapy. In particular, the present invention relates to the use of platelet-rich plasma (PRP) for predicting the responsiveness of a patient to cell therapy. The present invention also relates to methods of cell therapy that involve the use of PRP as an initial step in the treatment protocol.

### BACKGROUND OF THE INVENTION

Any discussion of the prior art throughout the specification should in no way be considered as an admission that such prior art is widely known or forms part of the common general knowledge in the field.

It has been previously shown that most patients with osteoarthritis respond positively to treatment with the stromal vascular fraction (SVF) of adipose tissue for pain and symptoms as measured by Western Ontario and McMaster Universities Osteoarthritis Index (WOMAC) scores (AU 2012902719). However, approximately 15% of patients do not respond in a positive manner to SVF treatment, in that they have no response or less than 15% improvement in their WOMAC scores.

There is a need for a simple test to determine suitability of a subject to undergo liposuction and SVF treatment for osteoarthritis. The test should also be suitable for determining whether a subject has osteoarthritis which may be treated with SVF, or stem cells or progenitor cells contained within the SVF.

It is an object of the present invention to overcome or ameliorate at least one of the disadvantages of the prior art, or to provide a useful alternative.

### SUMMARY OF THE INVENTION

Surprisingly, the present inventors have found that there is a positive correlation between patients that show a short term response to PRP administration by a reduction in pain and/or increase mobility and patients that respond to SVF administration for osteoarthritis.

According to one aspect, the present invention provides a method for predicting the responsiveness of a subject to cell therapy for osteoarthritis comprising administering platelet-rich plasma (PRP) to an affected joint of the subject and assessing pain and/or mobility in the joint, wherein a decrease in pain and/or an increase in mobility indicates that the subject will be responsive to cell therapy.

According to another aspect, the present invention provides a method of treating osteoarthritis in a subject comprising the steps of:
administering platelet-rich plasma (PRP) to an affected joint of the subject; and
assessing pain and/or mobility in the joint,

wherein if a decrease in pain and/or an increase in mobility is observed then the subject is administered a stromal vascular fraction and/or stein cells.

According to another aspect, the present invention provides platelet-rich plasma (PRP) for use in predicting the responsiveness of a subject to cell therapy for osteoarthritis, wherein a decrease in pain and/or an increase in mobility in an affected joint following administration of PRP to the joint indicates that the subject will be responsive to cell therapy.

According to another aspect, the present invention provides a stromal vascular fraction and/or stem cells for use in treating osteoarthritis in a subject, wherein the stromal vascular fraction and/or stem cells is administered to the subject if a decrease in pain and/or an increase in mobility is observed in an affected joint of the subject following administration of platelet-rich plasma (PRP) to the joint.

According to another aspect, the present invention provides use of platelet-rich plasma (PRP) for the manufacture of a medicament for predicting the responsiveness of a subject to cell therapy for osteoarthritis, wherein a decrease in pain and/or an increase in mobility in an affected joint of the subject following administration of the medicament to the joint indicates that the subject will be responsive to cell therapy.

According to another aspect, the present invention provides use of a stromal vascular fraction and/or stem cells for the manufacture of a medicament for treating osteoarthritis in a subject, wherein the medicament is administered to the subject if a decrease in pain and/or an increase in mobility is observed in an affected joint of the subject following administration of platelet-rich plasma (PRP) to the joint.

According to another aspect, the present invention provides a kit comprising:
platelet-rich plasma (PRP): and
a stromal vascular fraction and/or stem cells.

In a preferred embodiment, the cell therapy is administration of a stromal vascular fraction and/or stem cells.

In a preferred embodiment, the PRP is leukocyte-rich-PRP, PRP without leukocytes, degranulated PRP and/or platelet rich fibrin.

In a preferred embodiment, the stromal vascular fraction and/or stem cells are obtained from adipose tissue and/or bone marrow.

In a preferred embodiment, the stem cells are adult stem cells.

In a preferred embodiment, the stem cells are mesenchymal stem cells and/or mesenchymal precursor cells.

In a preferred embodiment, the stem cells are isolated, cultured and/or differentiated in vitro prior to injection.

In a preferred embodiment, the PRP is autologous PRP.

In a preferred embodiment, the PRP is allogeneic PRP.

In a preferred embodiment, the PRP is heterologous PRP.

In a preferred embodiment, about 1 ml to about 7 ml of PRP is administered to the subject. For example, about 1 ml to about 2 ml, about 1 ml to about 3 ml, about 1 ml to about 4 ml, about 1 ml to about 5 ml, about 1 ml to about 6 ml, about 2 ml to about 3 ml, about 2 ml to about 4 ml, about 2 ml to about 5 ml, about 2 ml to about 6 ml, about 2 ml to about 7 ml, about 3 ml to about 4 ml, about 3 ml to about 5 ml, about 3 ml to about 6 ml, about 3 ml to about 7 ml, about 4 ml to about 5 ml, about 4 ml to about 6 ml, about 4 ml to about 7 ml, about 5 ml to about 6 ml, about 5 ml to about 7 ml, about 6 ml to about 7 ml, about 1 ml, about 2 ml, about 3 ml, about 4 ml, about 5 ml, about 6 ml or about 7 ml of PRP may be administered to the subject.

In a preferred embodiment, the PRP is administered to the subject about 1 week to about 12 weeks prior to administration of SVF and/or stein cells. For example, the PRP may be administered to the subject about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 week, about 2 weeks, about 3 weeks, about 4 weeks, about 5 weeks, about 6 weeks, about 7 weeks, about 8 weeks, about 9 weeks, about 10 weeks, about 11 weeks or about 12 weeks prior to administration of SVF and/or stem cells.

In a preferred embodiment, the stromal vascular fraction and/or stem cells are administered intra-articularly.

In a preferred embodiment, the stromal vascular fraction and/or stem cells are administered intravenously.

In a preferred embodiment, the stromal vascular fraction and/or stem cells are administered intra-articularly and intravenously.

In a preferred embodiment, about 50 million to 500 million SVF cells are administered to the subject. For example, about 50 million to about 100 million SVF cells, about 50 million to about 200 million SVF cells, about 50 million to about 300 million SVF cells, about 50 million to about 400 million SVF cells, about 100 million to about 200 million SVF cells, about 100 million to about 300 million SVF cells, about 100 million to about 400 million SVF cells, about 100 million to about 500 million SVF cells, about 200 million to about 300 million SVF cells, about 200 million to about 400 million SVF cells, about 200 million to about 500 million SVF cells, about 300 million to about 400 million SVF cells, about 300 million to about 500 million SVF cells, about 400 million to about 500 million SVF cells, about 50 million SVF cells, about 100 million SVF cells, about 150 million SVF cells, about 200 million SVF cells, about 250 million SVF cells, about 300 million SVF cells, about 350 million SVF cells, about 400 million SVF cells, about 450 million SVF cells or about 500 million SVF cells may be administered to the subject.

In a preferred embodiment, the kit contains instructions for use in a method of treating osteoarthritis in a subject, the method comprising the steps of;
administering platelet-rich plasma (PRP) to an affected joint of the subject; and
assessing pain and/or mobility in the joint,

wherein if a decrease in pain and/or an increase in mobility is observed then a stromal vascular fraction and/or stem cells is administered to the subject.

In a preferred embodiment, the kit is used in a method of treating osteoarthritis in a subject, the method comprising the steps of;
administering platelet-rich plasma (PRP) to an affected joint of the subject; and
assessing pain and mobility in the joint,

wherein if a decrease in pain and/or an increase in mobility is observed then a stromal vascular fraction and/or stem cells is administered to the subject.

As used herein, the terms “platelet-rich plasma” or “PRP” refers to a blood fraction rich in platelets and their associated growth factors (i.e., plasma having more than the normal quantity of platelets found in whole blood).

Several different preparations of PRP may be useful in the invention including, but not limited to, PRP without leukocytes and leukocyte-rich-PRP (L-PRP), as well as platelet rich fibrin and degranulated PRP (Mazzocca at al., J Bone Joint Surg Am 2012; 94:308-316). These different preparations are essentially similar in that a mix of growth factors and cytokines are released from platelets in vitro or in vivo and are produced by well known means (Mazzocca at al., J Bone Joint Surg Am 2012; 94:308-316). There are many commercial PRP extraction kits readily available (Castillo at al., Am K Sports Med 2011; 39(2): 266 to 271).

Heterologous PRP may be obtained from any source, including commercial sources or blood banks.

As used herein, the term “adipose” refers to any fat tissue. The adipose tissue may be brown or white adipose tissue. The adipose tissue may be mesenchymal or stromal. Preferably, the adipose tissue is subcutaneous white adipose tissue. The adipose tissue may be from any organism having fat tissue. Preferably the adipose tissue is mammalian, most preferably the adipose tissue is human. A convenient source of human adipose tissue is that derived from liposuction surgery or other surgery. However, the source of adipose tissue or the method of isolation of adipose tissue is not critical to the invention.

As used herein the term “stromal vascular fraction” or “SVF” refers to a fraction, comprising cells, derived from blood vessels and surrounding tissue found in adipose tissue or bone marrow. The fraction may comprise different cell types including, by way of example, mesenchymal stem cells, early mesenchymal/stromal precursor cells, hematopoietic cells, hematopoietic stem cells, platelets, Kupffer cells, osteoclasts, megakaryocytes, granulocytes, NK cells, endothelial precursor or progenitor cells, CD34+ cells, Stro-1+ cells, Stro-3+ cells CD29+ cells, CD166+ cells, Thy-1+ or CD90+ stem cells, CD44+ cells, immune cells such as monocytes, leukocytes, lymphocytes, BandT cells, NK cells, macrophages, neutrophil leukocytes, neutrophils, neutrophil granulocytes, and the like. The stromal vascular fraction also includes cells expressing any of the markers or any combination thereof disclosed herein. As used herein, the term “stromal vascular fraction” includes within its scope mesenchymal vascular fractions, mesenchymal fractions, stromal fractions, and the like.

As used herein, the term “adult stem cell” refers to undifferentiated cells found throughout the body after embryonic development in children and adults that divide to replenish dying cells and regenerate damaged tissues. As used herein, the term “adult stem cell” excludes cells obtained from a foetus or an embryo.

As used herein, the term “differentiated” refers to a cell that has achieved a state of maturation such that the cell demonstrates biological specialization and/or adaptation to a specific environment and/or function. Typically, a differentiated cell is characterized by expression of genes that encode differentiation-associated proteins in that cell. For example expression of GALC in a leukocyte is a typical example of a differentiated leukocyte.

As used herein, the term “mesenchymal stem cell” refers to stromal or mesenchymal cells or early mesenchymal/stromal precursor cells which are multipotent and can serve as stem cell-like precursors to a variety of different cell types such as but not limited to adipocytes, osteocytes, chondrocytes, muscle and neuronal/glial cell lineages. Mesenchymal stem cells make up a subset population derivable from, for example, adipose tissue and bone marrow. As used herein, the term “mesenchymal stem cell” includes within its scope stromal stem cells, marrow stromal cells, multipotent stromal cells, mesenchymal precursor cells, and the like.

The terms “precursor cell”, “progenitor cell”, and “stein cell” are used interchangeably in the art and herein, and refer either to a pluripotent, or lineage-uncommitted, progenitor cell, which is potentially capable of an unlimited number of mitotic divisions to either renew itself or to produce progeny cells which will differentiate into the desired cell type.

As used herein, the term “multipotent”, “multipotential” or “multipotentiality” is meant to refer to the capability of a stem cell to differentiate into more than one type of cell.

As used herein, the term “heterologous” refers to any material derived from another individual.

As used herein, the term “allogeneic” refers to any material derived from another individual of the same species.

As used herein, the term “autologous” refers to any material derived from an individual and re-introduced to the same individual.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE FIGURES

FIG. 1: Correlation between responses to PRP and SVF in patients with osteoarthritis of the knee or hip.

### DETAILED DESCRIPTION OF THE PREFERRED EMBODIMENT

This invention is based on the surprising finding that there is a positive correlation between a patient's response to PRP administration and their response to SVF administration for osteoarthritis.

Briefly, PRP is prepared using well known techniques (Mazzocca et al., J Bone Joint Surg Am 2012; 94:308-316) and injected into the damaged site e.g., into the osteoarthritic knee, elbow, shoulder etc. Reduction in pain is measured pre- and post-treatment by WOMAC scores or similar osteoarthritis scoring sheets (Patel el al., Am J Sport Med 2013; 41: 356-364). If a reduction in pain or osteoarthritis symptoms is observed within 1 to 3 weeks of administration of PRP there is a surprisingly high chance that the patient will respond to SVF treatment for osteoarthritis.

The present invention will now be described in more detail with reference to specific but non-limiting examples describing specific compositions and methods of use. It is to be understood, however, that the detailed description of specific procedures, compositions and methods is included solely for the purpose of exemplifying the present invention. It should not be understood in any way as a restriction on the broad description of the inventive concept as set out above.

### EXAMPLES

### Example 1

### Preparation of PRP

1) 1−4×9 mL Acid citrate dextrose collection tubes were filled with blood to the black dot (vacuum pressure). The blood was drawn using an 18 G needle or larger to avoid activating the platelets by shearing. The contents of the blood tubes were mixed by inverting the tubes 3-4 times.
2) The blood-filled tubes were centrifuged at 450 g×10 min resulting in three layers—red blood cell layer, buffy coat layer and PRP layer (bottom to top).
3) For PRP without leukocytes, the PRP layer was removed from each tube with care taken not to disturb the buffy coat and platelet red blood cell layers.
4) For leukocyte-rich PRP, the PRP and huffy coat layers were removed with care taken not to remove the red blood cell layer.

### Example 2

### Preparation of Concentrated PRP

1) 1−4×9 mL Acid citrate dextrose collection tubes were filled with blood to the black dot (vacuum pressure). The blood was drawn using an 18 G needle or larger to avoid activating the platelets by shearing. The contents of the blood tubes were mixed by inverting the tubes 3-4 times.
2) The blood-filled tubes were centrifuged at 450 g×10 min resulting in three layers—red blood cell layer, buffy coat layer and PRP layer (bottom to top).
3) For PRP without leukocytes, the PRP layer was removed from each tube with care taken not to disturb the buffy coat and platelet red blood cell layers.
4) For leukocyte-rich PRP, the PRP and huffy coat layers were removed with care taken not to remove the red blood cell layer.
5) The PRP was combined and centrifuged for 2000 g/10 min—a small pellet of platelets at the bottom of the tube formed.
6) The top platelet poor plasma was removed with a transfer pipette down to 1.5 mL and discarded. The pellet was resuspended in the remaining 1.5 mL using the same transfer pipette.

### Example 3

### Preparation of Platelet-Rich Fibrin and Degranulated PRP

1) PRP or concentrated PRP produced according to examples 1 and 2 was clotted by adding calcium gluconate and mixing well—the tube may placed in a hot water bath (37° C.—without shaking) or left at room temperature for longer period of time.
2) The PRP formed a solid gel, which is platelet-rich fibrin.
3) The gel partially dissolves leaving a fluid which is degranulated PRP.

### Example 4

### Preparation of Adipose Tissue by Liposuction

An excess amount of Tumescent solution (containing, in one litre of normal saline, ling adrenalin, 800 mg lignocaine and 10 mLs of a 8.4% sodium bicarbonate solution), which exceeds the amount of liposuction to be aspirated prior to the liposuction operation, was infused into the hypodermic fat layer (tumescent method), and thereafter cannulae having, for example, 2-3 mm of inner diameter (made of metal with aspirator) was used for the liposuction operation. Liposuction operations are well known in the art and are described, for example, in Biyo Seikei Shujutsu Practice 2 (Cosmetic Operation Practice 2), ed. Masanari ICHIDA, Ryusaburo TANINO, and Yoshiaki HOSAKA, published by BUNKODO, pp. 429-469, which is incorporated herein by reference in its entirety.

Aspirated fat was washed with saline. About 50 ml to ten litres of washed aspirate may be generated, and the resultant adipose tissue derived cellular materials used for derivation of stromal vascular fractions.

### Example 5

### Preparation of Adipose Tissue by Surgery

Fat tissue was obtained by surgery from human subjects who had given their informed consent. Separation was conducted with techniques well known in the art. Briefly, human fat tissue was aseptically separated from fat tissue suctioned from human subjects who had given their informed consent. The resultant adipose tissue derived cellular materials were used for derivation of stromal vascular fractions.

### Example 6

### Preparation of a Stromal Vascular Fraction of Adipose Tissue by Collagenase Treatment

1) Between 50-1000 mL of lipoaspirate was obtained from the patient's abdomen using a 3 mm cannula and Modified Klein's solution. The lipoaspirate was rinsed with normal saline and placed in 500 mL centrifuge pots.
2) Collagenase (Serva) (filter sterilized through a 0.22 um sterile filter) was added to achieve a final concentration of 0.05%.
3) The sample was incubated at 37° C. for 30-90 minutes in a hot water bath, and gently agitated. During the incubation the sample was gently inverted by hand every 15 minutes.
4) Following incubation the sample was centrifuged at 500 g×5 min Three layers were present after the centrifugation. The top yellow/clear layer (lipid layer). The white fibrous middle layer and, the red/white bottom layer which has a cell pellet at the bottom of the tube.
5) The cell pellets were removed from the pots by drawing up the pellet with a mixing cannula and syringe.
6) The cell pellets were expelled into a 50 mL centrifuge tube and PBS added to 40 mL. The tube contents were aseptically filtered through a 100 um steriflip (Millipore) using a vacuum pump into the 50 mL, tube.
7) The filtrate was centrifuged at 500 g for 5 minutes.
8) The supernatant was removed without disturbing the pellet and all cell pellets combined into the one centrifuge tube. The resultant pellet was resuspended and 40 mL PBS added.
9) The cell suspension was centrifuged at 500 g for 5 minutes and the supernatant removed.
10) 20 mL PBS was added and the cell suspension filtered through a 60 μm steriflip (Millipore).
11) The filtrate was centrifuged at 500 g for 5 minutes and the supernatant removed. A sample was removed for cell counting (a sample of 50 μl of well mixed cells was added to 0.4% of trypan blue, mixed and allowed to stand for 1-2 minutes before placing the sample into a chamber of the haemocytometer. Cell count and viability was determined by counting at least 100 cells in the grid area. Viable cells were determined by exclusion of trypan blue).
12) 5-10 mL platelet-rich plasma or normal saline was added.
13) The sample was drawn up into a syringe and injected into normal saline IV 1 litre hag for infusion into the patient.

### Example 7

### Preparation of a Stromal Vascular Fraction Comprising Viable Stem Cells by Ultrasonic Cavitation Using 25 ml of Lipoaspirate

1) Adipose tissue was derived from liposuction aspirates and 25 ml of aspirate was placed into 50 ml centrifuge tubes.
2) Excess fluid was removed by centrifugation at 200 g/2 minutes to separate out the excess fluid and adipose tissue. The excess fluid at the base of the tube was removed, typically leaving 20 ml of adipose.
3) The ultrasonic cavitation device probe was placed into the adipose tissue and the amplitude set at 50%, cycle 0.4. The probe was raised and lowered for 1 minute and then for 30 seconds at the top for each tube.
4) After ultrasonication a thick solution was observed in the tube and the tissue centrifuged at 800 g/5 min.
5) After centrifugation there were 3 layers—the top lipid layer, the middle floating layer containing extracellular matrix and stromal vascular cells, and a bottom layer of fluid.
6) The top lipid layer was removed and discarded using a mixing cannula and syringe (removal of the lipid layer permits a separation of cells when isotonic fluid is added) and the remaining contents of the tube were mixed well to further disrupt the extra-cellular matrix.
7) An isotonic solution (typically 0.9% saline) was added to the tube to 45 ml and the mix centrifuged at 800 g/5 mins initiating the cells and extra-cellular matrix to fall out and pellet at the bottom.
8) A large pellet was observed at the bottom of tube containing extracellular matrix and the stromal vascular fraction comprising viable and functional stem cells. Excess adipose was at the top and saline in the middle.
9) The top layer of excess adipose and saline was removed above the pellet until approximately 15 ml of fluid was left above the pellet. The cell pellet was vortexed and filtered through a 100 um filter to remove any large debris.
10) The cell solution was used as is, or further concentrated by centrifugation and removal of excess fluid, or combining multiple samples.
11) A sample was removed for cell counting and viability to ascertain quantity of cells to be administered to the patient.

### Example 8

### Preparation of a Stromal Vascular Fraction Comprising Viable Stem Cells by Ultrasonic Cavitation Using 45 ml of Lipoaspirate

1) Adipose tissue was derived from liposuction aspirates and 45 ml of aspirate was placed into 50 ml centrifuge tubes
2) Excess fluid was removed by centrifugation at 200 g/2 minutes to separate out the excess fluid and adipose tissue. The excess fluid at the base of the tube was removed, typically leaving 40 ml of adipose.
3) The ultrasonic cavitation device probe was placed into the adipose tissue and the amplitude set at 50%, cycle 0.4. The probe was raised and lowered for 90 seconds and then for 30 seconds 20 ml mark on the tube and then for 30 seconds at the top of each tube.
4) After ultrasonication a thick solution was observed in the tube and the tissue centrifuged at 800 g/5 min.
5) After centrifugation there were 3 layers—the top lipid layer, the middle floating layer containing extracellular matrix and stromal vascular cells, and a bottom layer of fluid.
6) The top lipid layer was removed and discarded using a mixing cannula and syringe (removal of the lipid layer permits a separation of cells when isotonic fluid is added) and the remaining contents of the tube mixed well to further disrupt the extra-cellular matrix.
7) An isotonic solution (typically 0.9% saline) was added to the tube to 45 ml and the mix centrifuged at 800 g/5 mins initiating the cells and extra-cellular matrix to fall out and pellet at the bottom.
8) A large pellet was observed at the bottom of tube containing extracellular matrix and the stromal vascular traction comprising viable and functional stem cells. Excess adipose was at the top and saline in the middle.
9) The top layer of excess adipose and saline was removed above the pellet until approximately 15 ml of fluid was left above the pellet. The cell pellet was vortexed and filtered through a 100 um filter to remove any large debris.
10) The cell solution was used as is, or further concentrated by centrifugation and removal of excess fluid, or combining multiple samples.
11) A sample was removed for cell counting and viability to ascertain quantity of cells to be administered to the patient.

### Example 9

### Preparation of Bone Marrow

Bone marrow was extracted from the sternum, posterior ilium, or anterior ilium using established techniques. Briefly, the site was prepared with Betadine solution and local anaesthesia was placed under the skin. A longer needle was used to identify the midpoint of the iliac crest and deposit 3-4 mL 2% Xylocaine under the periosteum. A “J” needle was inserted into the anterior/posterior iliac wing. The needle was rotated gently into 1 cm of the marrow cavity. The stylet was removed from the needle and a 5-cc syringe attached. Bone marrow was aspirated by retraction of the plunger of the syringe. After 2-3 mL of marrow was collected, the needle was repositioned if more marrow could be obtained.

Bone marrow cells harvested by the perfusion or aspiration method were centrifuged and suspended in 15 mL of PBS. The cells were placed on 15 mL of Lymphoprep density solution (1.077 g/mL). After centrifugation for 30 minutes at 2,000 rpm at room temperature, the bone marrow cells were collected from the defined layer at the interface.

### Example 10

### Preparation of Expanded Mesenchymal Stem Cells

Adult stem cell were be obtained from adipose tissue or bone marrow by any suitable method and cultured without differentiation using standard cell culture medium (e.g., alphaMEM typically supplemented with foetal calf serum, human serum or serum free medium). Primary cultures were plated at 1×10⁶/100 mm. The cells were expanded for 1-2 passages (but can be passaged up to 7 times) in 5% CO, or hypoxic environment. Such cells may be clonally passaged if required. The isolated autologous or allogeneic cells were cultured to a suitable point and viability and yield assessed by standard methods. These cells may then be injected intra-articularly or intravenously.

### Example 11

### Administration of PRP and SVF to Patients

Patients presented with osteoarthritis of the hips or knees.

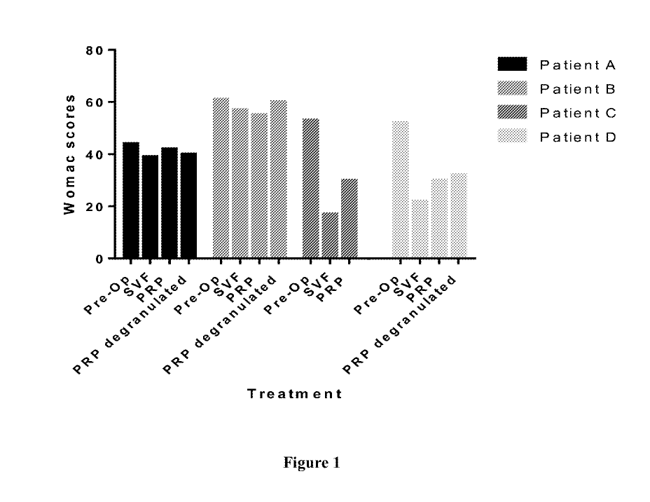

Patient A was injected with SVF in the knee but showed no improvement (reduction in pain) after 3 months. PRP without leukocytes was injected into the knee 5 months after SVF injection and degranulated PRP was injected into the knee 5 months after PRP without leukocytes injection. Administration of PRP and degranulated PRP did not result in a reduction in pain (Table 1 and FIG. 1).

Patients B and D were administered PRP without leukocytes intra-articularly and 4 weeks later were administered degranulated PRP intra-articularly. Four weeks after administration of degranulated PRP, the patients were administered SVF intra-articularly and intravenously. Patient C was administered PRP without leukocytes intra-articularly and 4 weeks later was administered SVF intra-articularly.

Patient B showed no reduction in pain following PRP administration or SWF administration (Table 1 and FIG. 1). Patients C and D showed reduction in pain following both PRP administration and SVF administration (Table 1 and FIG. 1).

TABLE 1

| | PRP | Degranulated | SVF | | PRP pain | SVF pain |
|---|---|---|---|---|---|---|
| | (ml) | PRP (ml) | (10⁶) | location | reduction | reduction |
| Patient A | 5 | 3 | 125 | knee | No | No |
| Patient B | 4 | 3 | 109 | hip | No | No |
| Patient C | 5 | — | 87 | knee | Yes | Yes |
| Patient D | 6 | 3 | 100 | knee | Yes | Yes |

The correlation shown above has been confirmed in 28 additional patients. All patients who reported a decrease in pain and/or increase in mobility following PRP administration (as measured by WOMAC score) responded to SVF administration.

1. A method for predicting the responsiveness of a subject to cell therapy for osteoarthritis comprising administering platelet-rich plasma (PRP) to an affected joint of the subject and assessing pain and/or mobility in the joint, wherein a decrease in pain and/or an increase in mobility indicates that the subject will be responsive to cell therapy.

2. The method according to claim 1 wherein the cell therapy is administration of a stromal vascular fraction and/or stem cells.

3-14. (canceled)

15. The method according to claim 1 wherein the PRP is leukocyte-rich-PRP, PRP without leukocytes, degranulated PRP and/or platelet rich fibrin.

16. The method according to claim 2 wherein the stromal vascular fraction and/or stem cells are administered intra-articularly and/or intravenously.

17. The method according to claim 2 wherein the stromal vascular fraction and/or stems cells are obtained from adipose tissue.

18. The method according to claim 2 wherein the stem cells are adult stem cells.

19. The method according to claim 2 wherein the stem cells are mesenchymal stem cells and/or mesenchymal precursor cells.

20. The method according to claim 2 wherein the stem cells are isolated, cultured and/or differentiated in vitro prior to administration.

21. The method according to claim 1 wherein the PRP is autologous PRP.

22. The method according to claim 1 wherein the PRP is allogeneic PRP.

23. The method according to claim 1 wherein about 1 ml to about 7 ml of PRP is administered to the subject.

24. The method according to claim 2 wherein the PRP is administered to the subject about 1 week to about 12 weeks prior to administration of SVF and/or stem cells.

25. The method according to claim 2 wherein about 50 million to about 500 million SVF cells are administered to the subject.

26. A method of treating osteoarthritis in a subject comprising the steps of:
administering platelet-rich plasma (PRP) to an affected joint of the subject; and
assessing pain and/or mobility in the joint,
wherein if a decrease in pain and/or an increase in mobility is observed then the subject is administered a stromal vascular fraction and/or stem cells.

27. The method according to claim 26 wherein the PRP is leukocyte-rich-PRP, PRP without leukocytes, degranulated PRP and/or platelet rich fibrin.

28. The method according claim 26 wherein the stromal vascular fraction and/or stem cells are administered intra-articularly and/or intravenously.

29. The method according to claim 26 wherein the stromal vascular fraction and/or stems cells are obtained from adipose tissue.

30. The method according to claim 26 wherein the stem cells are adult stem cells.

31. The method according to claim 26 wherein the stem cells are mesenchymal stem cells and/or mesenchymal precursor cells.

32. The method according to claim 26 wherein the stem cells are isolated, cultured and/or differentiated in vitro prior to administration.

33. The method according to claim 26 wherein the PRP is autologous PRP.

34. The method according to claim 26 wherein the PRP is allogeneic PRP.

35. The method according to claim 26 wherein about 1 ml to about 7 ml of PRP is administered to the subject.

36. The method according to claim 26 wherein the PRP is administered to the subject about 1 week to about 12 weeks prior to administration of SVF and/or stem cells.

37. The method according to claim 26 wherein about 50 million to about 500 million SVF cells are administered to the subject.
